(19) United States
(12) Patent Application Publication
(10) Pub. No.: US 2026/0128600 A1
(43) Pub. Date: May 7, 2026

(54) NEONATAL POD WITH CHARGER

(71) Applicant: GE Precision Healthcare LLC, Waukesha, WI (US)

(72) Inventor: Sudeepa Aithala, Bengaluru (IN)

(73) Assignee: GE Precision Healthcare LLC, Waukesha, WI (US)

(21) Appl. No.: 18/938,592

(22) Filed: Nov. 6, 2024

(51) Int. Cl. H02J 7/35; A61B 5/00; A61B 5/0205; A61G 11/00

(52) U.S. Cl. CPC H02J 7/35; A61B 5/002; A61B 5/02055; A61B 2503/045; A61G 11/00

(57) ABSTRACT

A neonatal pod with charger includes a physical connection configured to couple to a neonatal patch that is configured to measure signals from a patient. The neonatal pod with charger also includes a wireless communication interface, a rechargeable battery, and a battery charger. The battery charger is configured to convert ambient radiation into an electrical current and charge the rechargeable battery using the electrical current.

### BACKGROUND

The present disclosure generally relates to a neonatal pod, and more particularly to systems and methods for a neonatal pod with charger.

Surface electrodes, adhered to the surface of a patient's skin, enable electrical contact between the patient's skin and a conductor. Additionally, the conductor may connect the surface electrodes to a sensor that is monitoring a physiological condition of that patient. More specifically, the surface electrodes may conduct potentials from the patient's body, e.g., skin temperature, and enable measurement of the potentials. This physiological monitoring may be useful for tracking temperature, respiration rates, pulse rates, producing electrocardiograms (ECGs), producing electroencephalograms (EEGs), and the like.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

A neonatal pod with charger includes a physical connection configured to couple to a neonatal patch that is configured to measure signals from a patient. The neonatal pod with charger also includes a wireless communication interface, a rechargeable battery, and a battery charger. The battery charger is configured to convert ambient radiation into an electrical current and charge the rechargeable battery using the electrical current.

In one embodiment, the battery charger comprises a solar charger.

In one embodiment, the battery charger comprises a thermal charger.

In one embodiment, the thermal charger converts thermal energy from a body of the patient to electrical current.

In one embodiment, the neonatal pod with charger includes a processing device and a memory device configured to store instructions. The instructions are executable by the processing device to use the wireless communication interface to provide the signals over a network to a base station configured to make determinations about the health of the patient based on the signals.

In one embodiment, the instructions are executable by the processing device to determine that the wireless communication interface is not connected to the network. Additionally, the instructions are executable by the processing device to store the signals in the memory device until the wireless communication interface is re-connected to the network.

In one embodiment, the neonatal pod with charger includes an alarm device. Further, the instructions are executable by the processing device to activate the alarm in response to determining that the wireless communication interface is not connected to the network.

In one embodiment, the instructions are executable by the processing device to determine a type of the neonatal patch.

In one embodiment, the signals represent a physiological condition of the patient.

In one embodiment, the patch is selected from a group consisting of an electrocardiogram patch, a core temperature patch, a peripheral temperature patch, a respiratory rate patch, a pulse rate patch, and a specific percentage of oxygen patch.

A neonatal pod with charger includes a processing device, a memory device configured to store instructions executable by the processing device, a physical connection configured to couple to a neonatal patch that is configured to measure signals from a patient, a wireless communication interface, a rechargeable battery, and a battery charger that is configured to convert ambient radiation into an electrical current, and charge the rechargeable battery using the electrical current.

In one embodiment, the battery charger comprises a solar charger.

In one embodiment, the battery charger comprises a thermal charger.

In one embodiment, the thermal charger converts thermal energy from a body of the patient to electrical current.

In one embodiment, the instructions are executable by the processor to use the wireless communication interface to provide the signals over a network to a base station configured to make determinations about the health of the patient based on the signals.

In one embodiment, the instructions are executable by the processing device to determine that the wireless communication interface is not connected to the network. Additionally, the instructions are executable by the processing device to store the signals in the memory device until the wireless communication interface is re-connected to the network.

In one embodiment, the neonatal pod with charger includes an alarm device.

Additionally, the instructions are executable by the processing device to activate the alarm in response to determining that the wireless communication interface is not connected to the network.

In one embodiment, the instructions are executable by the processing device to determine a type of the neonatal patch.

In one embodiment, the signals represent a physiological condition of the patient.

In one embodiment, the patch is selected from a group consisting of an electrocardiogram patch, a core temperature patch, a peripheral temperature patch, a respiratory rate patch, a pulse rate patch, and a specific percentage of oxygen patch.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a perspective view of a neonatal care system, example neonate pod with charger, and patch according to one embodiment of the present disclosure.

FIG. 2 is a perspective view of a neonatal care system, example neonate pod with charger, and patch according to one embodiment of the present disclosure.

FIG. 3 is a diagram of a neonate, example neonate pod with charger, chest patch, and extremity patch according to one embodiment of the present disclosure.

FIG. 4A is a top view of an example neonate pod with charger and patch according to one embodiment of the present disclosure.

FIG. 4B is a top view of an example neonate pod with charger and patch according to one embodiment of the present disclosure.

FIG. 4C is a top view of an example neonate pod with charger and patch according to one embodiment of the present disclosure.

FIG. 5 is a block diagram an example neonate pod with charger and patch according to one embodiment of the present disclosure.

FIG. 6 is a diagram of a system for an example neonate pod with charger and patch according to one embodiment of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally, or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The inventors have recognized problems with current neonatal patches, which may be used to measure various physiological conditions of a neonate. For example, neonatal patches may include a number of wires that communicate signals from the patch to a processing device, which may make the physiological measurements. However, these wires can impede access to, and thus, care for, the neonate. Additionally, neonatal patches may connect with devices, such as pods, which may provide communications between the neonatal patch and the processing device performing the physiological measurements. Such pods may include a rechargeable battery power source. As such, a caretaker or other operator may periodically remove the pod from the neonatal patch in order to recharge the battery. However, removing the pod may stop the physiological monitoring because the pod is no longer available to communicate signals from the neonatal patch to the processing device. In some cases, recharging the battery may involve a multi-hour process, which means the neonate's physiological conditions may go unmonitored for several hours. In order to maintain the physiological monitoring, it may be possible to use a replacement pod during recharging. However, providing a replacement pod incurs an additional cost. Further, due to their relatively small size, and potentially fragile condition, neonates may be adverse to undue pressure on their bodies. However, the pod may include components that increase the weight of the pod. For example, the pod may include magnets, which may be useful for securing the pod to the neonatal patch. Further, the pod may include inductive elements that connect with a wireless charger that recharges the battery. Such components may increase the weight of the pod, and thus, pressure of the pod on the neonate's body. Additionally, in order to provide a relatively longer operating time, these pods may include relatively large, and thus, weighty batteries. In these ways, current neonatal patches and pods may interfere with the care of neonates, and add to their discomfort.

In view of the foregoing problems and challenges recognized by the inventors through their extensive research and experience in the field of neonatal care systems, the inventors have developed the disclosed improved pod, which may provide a wireless connection between a neonatal patch and a processing device. Further, such a pod may include a mechanical connection (e.g., a physical locking mechanism) to the neonatal patch, which may reduce the weight of the pod compared to current pods that use magnets to secure a pod to the neonatal patch. Additionally, according to some embodiments of the present disclosure, the pod may include one or more chargers that uses ambient light, ambient thermal energy (from the air, neonate care system warmer, and/or body of the neonate), solar energy, and the like, to charge the pod's battery. Having such charger(s) makes it possible to reduce the size of the pod's battery without reducing the length of operating time of the pod. Further, including such charger(s) may make it possible to reduce the battery size, and provide a lengthier operating period than that of a larger, and heavier, battery. Additionally, including the charger(s) means the weight of the pod may be reduced by eliminating inductive charging elements from the pod. Further, the pod may be reusable for numerous patch types, which may increase the usefulness of the pod. In these ways, some embodiments of the present disclosure may provide a pod that is more comfortable for the neonate, and more convenient for caretakers, while increasing operating times and utility, in comparison to current devices.

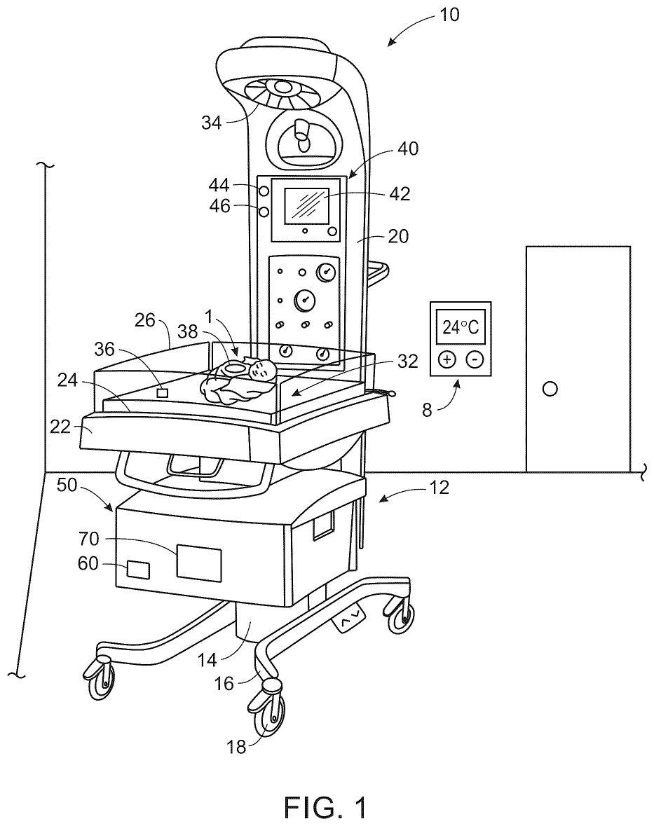

FIG. 1 is a perspective view of a neonatal care system 10, example neonate pod with charger 38, and patch according to one embodiment of the present disclosure. The neonatal care system 10 is shown within a room, such as a labor and delivery suite, or a neonatal intensive care unit, within a medical facility. The ambient air temperature within the room is controlled by room thermostat 8, which is adjustable up and down according to the specification of the patient and medical personnel in a customary manner. As described above, the neonatal care system 10 includes a number of sensing devices, operational components and displays whose functions one or more processing devices coordinate to enable the neonatal care system 10 to operate as described herein. For example, the neonate pod with charger 38 may receive signals from a disposable patch (not shown) that a caretaker may secure to the body of the patient 1. Such patches may have electrodes that conduct potentials from the patient's body that are indicative of heart and respiratory rates, temperature, fluid loss, and various other physiological conditions. Additionally, it may be possible for the controller to predict when the patient may be discharged based on these physiological conditions. Patches are described in greater detail below. According to some embodiments of the present disclosure, these electrodes may conduct potentials and generate signals for the neonate pod with charger 38. Additionally, the neonate pod with charger 38 may perform housekeeping processes on the data, and provide this data for another system(s), e.g., processing device), which may determine the physiological measurements, and use these measurements to make determinations about the patient's health and well-being. Further, the neonate pod with charger 38 may include a wireless communication interface (not shown), and a battery (not shown) that powers the processor, wireless communication interface, and other elements of the neonate pod with charger 38.

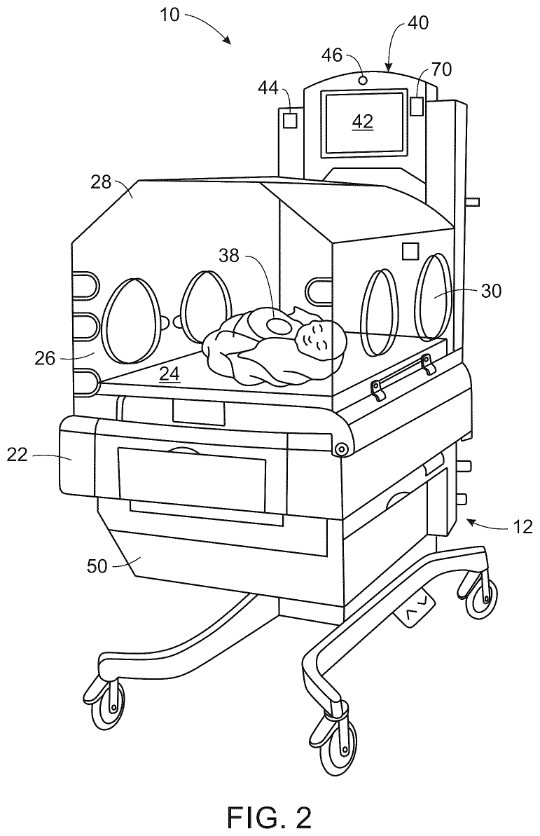

The neonatal care system 10 shown here is an infant warmer having some elements similar to the Giraffe® warmer produced by GE Healthcare™. In other embodiments, the neonatal care system 10 may be similar to an alternative infant warmer. The neonatal care system 10 includes a stand 12 supported by legs 14 and feet 16 provided with wheels 18 in a manner presently known in the art. The walls 26, and in certain cases a cover 28 (See FIG. 2), generally surround and cover the bed 24, to prevent the patient 1 from falling from the bed 24 and also to maintain a controlled environment within the interior. The air within the interior defined by the walls 26 (and when present, the cover 28) is also referred to as inside air 32. The temperature of the inside air 32 is controlled at least in part by operation of a heater 34. The heater 34 may be a heat generating device such as those used within the exemplary warmers described above. It should be recognized that when no cover 28 is present, the inside air 32 interior is more able to mix with the ambient air within the room. Additionally, the stand 12 also supports an enclosure 50, and contains a controller 70 (e.g., one or more microprocessors, computer processing devices, and the like) for operating the neonatal care system 10 in a manner presently known in the art. Additionally, the neonate pod with charger 38 may provide data that the controller 70 may process to make determinations about the physiology of the patient 1. A column 20 extends upwardly from the stand 12 and supports a platform 22. The platform 22 may be height adjustable along the column 20 in a manner presently known in the art.

The neonatal care system 10 further includes a user interface 40, which may include a display 42 configured to provide warning indications (text, colors, icons, and the like) as well as messages relating to operation of the neonatal care system 10. Additionally, the user interface 40 may include a speaker 44 and one or more lights 46. The speaker 44 and lights 46 may provide further information regarding the operational status of the neonatal care system 10 with integrated weighting. According to one embodiment of the present disclosure, the scale manager may provide the data feed of measured patient weights for presentation on the display 42. For example, the display 42 may show a graph indicating the change in measured patient weight over time.

Additionally, the speaker 44 and lights 46 may communicate information to a caretaker and/or operator via sounds, spoken text, spoken words, flashing, varying colors, and/or the lights being on or off. In this manner, as is discussed further below, the user interface 40 provides feedback customary of infant care systems 10 presently known in the art, but also additional information, warnings, and/or the like according to the present disclosure. It should be recognized that the user interface 40 may also or alternatively be provided via an external device (e.g., a mobile device such as a tablet or smart phone) in communication with the neonatal care system 10. For example, a smart phone may serve as the display 42, speaker 44, and/or lights 46 (alone or in conjunction with another display 42, speaker 44, and lights 46, on the neonatal care system 10) that communicates with the neonatal care system 10 via Bluetooth® or another wireless protocol known in the art.

As stated previously, one or more controllers 70 may use the signals provided by the neonate pod with charger 38 to determine the patient's physiological measurements, and use these measurements to make determinations about the patient's health and well-being. Accordingly, these controllers 70 may operate the display 42, speaker 44, and lights 46, to provide a warning or other information about the patient that prompts a caretaker or other operator to attend to the patient 1. Additionally, in the event of a break in communication between the controller 70 and neonate pod with charger 38, the controller 70 may operate the display 42, speaker 44, and lights 46 to sound alerts, provide communication network status, and/or prompt the caretaker to monitor the physiological measurements until the communication is restored. Further, the neonate pod with charger 38 may store signals from the patch in a local buffer that can store 2 or more hours of data. In this way, when communication is restored, the neonate pod with charger 38 may provide the data captured during the communication break, e.g., buffered data, to the controller 70 after communication is restored. The size of the buffer may vary, capable of storing hours of data. Additionally, the neonate pod with charger 38 may monitor predetermined physiological measurements such as, temperature and heart rate to identify emergency conditions. For example, the neonate pod with charger 38 may generate an alert, (e.g., send a signal to the controller 70 to operate the display 42, speaker 44, and/or lights 46) if the neonate's heart rate, respiratory rate, temperature, and the like, drop below a predetermined threshold. Alternatively, if such an emergency condition occurs while communication is lost between the neonate pod with charger 38 and the controller 70, the neonate pod with charger 38 may generate an alert for a caretaker (e.g., sound an alarm, flash lights, and the like).

Further, according to some embodiments of the present disclosure, the charger(s) of the neonate pod with charger 38 may capture ambient energy from the local environment (e.g., the room, neonatal care station 10, the neonate, and the like), and store at least a portion of the captured energy in the battery. In this way, the neonate pod with charger 38 may prolong the life of the neonate pod with charger's battery. By prolonging the life of the battery with ambient energy capture, it may be possible to use a smaller battery in the neonate pod with charger 38 than in current neonate pods. In some embodiments of the present disclosure, the charger(s) may include a thermal capture device that converts heat and/or heat differentials (produced by the heater 34, the ambient air, the neonate's body, and the like) into electric signals for storage in the neonate pod with charger's battery. In another example, the neonate care station 10 may include one or more light sources (not depicted) in the canopy 20, for example, to control the light in the patient's environment. Accordingly, the neonate pod with charger 38 may include a photovoltaic cell, or other photon-sensitive device, that converts the light from these light sources, ambient sunlight, and the like, into electrical signals for storage in the neonate pod with charger's battery.

FIG. 2 is a perspective view of a neonatal care system 10, example neonate pod with charger 38, and patch according to one embodiment of the present disclosure. In this example, the neonatal care system 10 is similar to that of FIG. 1, but as an incubator rather than an infant warmer. Similar to FIG. 1, the neonatal care system 10 of FIG. 2 includes stand 12, platform 22, bed 24, walls 26, user interface 40, image sensor 48, enclosure 50, and controller 70. Additionally, the neonatal care system 10 of FIG. 2 includes a cover 28, whereby the interior of the neonatal care system 10 is defined by the walls 26 and the cover 28. Further, the incubator of FIG. 2 includes portholes 30 within the walls 26 and/or cover 28 to provide access to the interior (e.g., patient 1, neonate pod with charger 38, neonatal patches (not shown), bed 24, and/or the platform 22) without opening one or more of the walls 26 and/or the cover 28 in a manner presently known in the art.

As stated previously, the neonate pod with charger 38 may receive signals from a disposable patch (not shown) that a caretaker may secure to the body of the patient 1. Such patches may have electrodes that conduct potentials from the patient's body that are indicative of heart and respiratory rates, temperature, and various other physiological conditions. Patches are described in greater detail below. According to some embodiments of the present disclosure, these electrodes may conduct potentials and generate signals for the neonate pod with charger 38. Additionally, the neonate pod with charger 38 may perform housekeeping processes on the data, and provide this data for another system(s), e.g., processing device), which may determine the physiological measurements, and use these measurements to make determinations about the patient's health and well-being. Further, the neonate pod with charger 38 may include a wireless communication interface (not shown), and a battery (not shown) that powers the processor, wireless communication interface, and other elements of the neonate pod with charger 38.

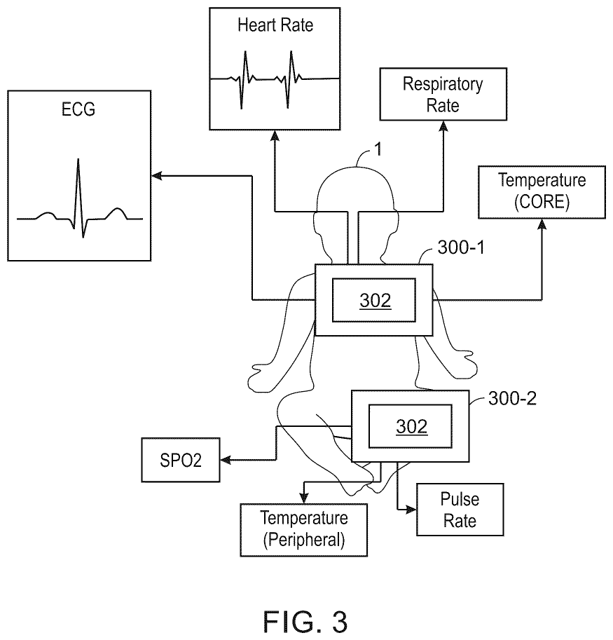

FIG. 3 is a diagram of a neonate (e.g., patient 1), example neonate pod with charger 302, chest patch 300-1, and extremity patch 300-2 (collectively referred to as patches 300) according to one embodiment of the present disclosure. As stated previously, the electrodes of a neonatal patch, such as the chest patch 300-1, may measure data useful for an echocardiogram (ECG), measure heart rate, respiratory rate, core temperature, fluid loss, and the like. Core temperature may refer to the temperature in the area approximating the upper abdomen of the patient's body. Fluid loss may refer to measuring the amount of sweat given out by the infant. The amount of sweat loss can also be used as a closed loop mechanism to increase the humidity inside the incubator.

Additionally, the extremity patch 300-2 may be located on a limb or successive extremity of the patient's limb. The extremity patch 300-2 may measure the patient's specific percentage of oxygen, peripheral temperature, pulse rate, fluid loss (through sweat) and the like. Beyond these examples, the patches 300 may provide measures of various other physiological and/or ambient states capable of being detected by conducting potentials from the body of the patient 1 via the patch's electrodes. Further, the size of the patch 300 may be relatively small, to accommodate the small body size of the neonate. Additionally, the size of the patch 300 may vary based on the type. For example, an ECG patch may be approximately 3 centimeters (cm) by 3 cm; a temperature patch may be 2 cm by 2 cm; and, an extremity patch may be 3 cm by 4 cm, or 3 cm by 5 cm.

According to some embodiments of the present disclosure, the neonate pod with charger 302 may receive signals from the patch, perform housekeeping processes on the data, and provide this data for another system(s), e.g., controller 70), which may determine the physiological measurements, and use these measurements to make determinations about the patient's health and well-being. Further, the neonate pod with charger 302 may include a wireless communication interface (not shown), and a battery (not shown) that powers the processor, wireless communication interface, and other electrically-powered elements of the neonate pod with charger 302. In some examples, the battery may be lithium-ion, phosphate, cadmium based, and the like. Such a battery may have a capacity of 500 milliamperes or more. Additionally, such a battery may have a weight approximating 25 grams (or less).

Further, the neonate pod with charger 302 may include a locking mechanism (not shown) to physically secure the neonate pod with charger 302 to, and be configured to electrically connect with, various types of patches 300. The connection may include a single or multi-pin connector. Additionally, the neonate pod with charger 302 may automatically detect the type of patch 300 with which the neonate pod with charger 302 is connected. According to some embodiments of the present disclosure, the neonate pod with charger 302 may determine the patch type based on the type of connection provided by the patch 300, the number of pins in the connection, the specific pins that connect (e.g., provide signals) from the patch 300, near-field communication signals from the patch 300, and the like. In this example, the type of patch 300 may refer to the type of data measured (e.g., SPO2, ECG, temperature, and the like) by the connected patch 300. Accordingly, the neonate pod with charger 302 may process the received data based on the determined patch type. Further, the neonate pod with charger 302 may transmit this data wirelessly to the controller 70. Additionally, the neonate pod with charger 302 may determine if there is a fault in the patch 300. For example, if the leads of the patch 300 are cut or otherwise non-functional, the neonate pod with charger 302 may identify such an issue, and signal an alert to the controller 70. Accordingly, the controller 70 may generate an alert for the caretaker or other operator to replace the patch 300.

In these ways, the neonate pod with charger 302 may reduce the number of wires connected to the patient 1, e.g., infant, to measure various parameters. Further, reducing the number of wires may improve access to the infant and interior of the neonate care station 10, for a caretake or other operator. According to some embodiments, the neonate pod with charger 302 may be re-usable pod, and placed on a disposable patch (e.g., patches 300) attached to the infant. Additionally, the neonate pod with charger 302 may wirelessly transmit the data to the controller 70.

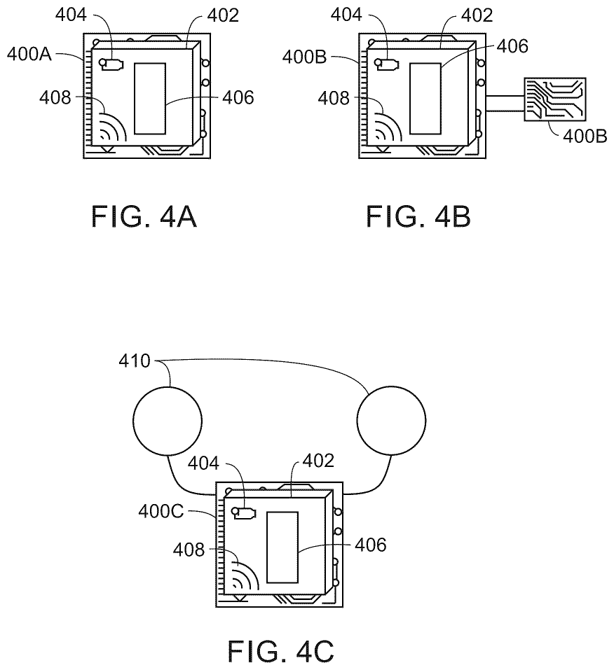

FIG. 4A is a top view of an example neonate pod with charger 402 and patch 400A according to one embodiment of the present disclosure. The patch 400A may be a chest patch, similar to the chest patch 300-1 described with respect to FIG. 3. In this example, the patch 400A may be useful for determining the core temperature of the patient 1.

The neonate pod with charger 402 may include a battery 404, charger 406, and a wireless communication interface 408. The battery 404 may be a power store that the neonate pod with charger 402 uses as a power source. The wireless communication interface 408 may be a computer network communication device that sends signals to, and receives signals from, the controller 70. Additionally, the neonate pod with charger 402 is connected to the patch 400A which provides signals indicative of the patient's core temperature. Accordingly, the neonate pod with charger 402 may send these signals to the controller 70 for processing.

According to some embodiments of the present disclosure, the neonate pod with charger 42 may include a charger 406. The charger 406 may be a transducer, such as a solar charger, that converts light, heat or other energy, into electrical current. This converted light, heat, or other energy may include radiation from ambient light, and/or an infrared warmer of the neonate care station 10. Further, the neonate pod with charger 402 may use this current to charge the battery 404. By charging the battery 404 in this way, the neonate pod with charger 402 may increase the amount of time for monitoring core temperature. Additionally, the charger 402 makes it possible for the battery 404 to be of a smaller size, and hence lighter weight than batteries in current pod devices. In this way, the neonatal pod with charger 402 can provide an improvement over the current devices, whose larger and heavier batteries may cause the patient 1 discomfort.

FIG. 4B is a top view of an example neonate pod with charger 402 and patch 400B according to one embodiment of the present disclosure. The patch 400A may be an extremity patch, similar to the extremity patch 300-2 described with respect to FIG. 3. In this example, the patch 400A may be useful for determining the peripheral temperature, specific percentage of oxygen saturation, heart rate, pulse rate, and/or the fluid loss of the patient 1. In this example, the neonate pod with charger 402 is connected to the patch 400B which provides signals indicative of the physiological conditions described above. Accordingly, the neonate pod with charger 402 may send these signals to the controller 70 for processing.

FIG. 4C is a top view of an example neonate pod with charger 402 and patch 400C according to one embodiment of the present disclosure. The patch 400C may be a chest patch, similar to the extremity patch 300-1 described with respect to FIG. 3. More specifically, the patch 400C may be an electrocardiogram (ECG) patch. Accordingly, the patch 400C may monitor heart activity by detecting electrical signals of the patient's heart. Additionally, the patch 400C may be useful for determining the core temperature, respiratory rate, specific percentage of oxygen saturation, pulse rate, bodily movement, sleep activity, sleep apnea, fluid loss, and the like, of the patient 1. In this example, the neonate pod with charger 402 is connected to the patch 400C which provides signals indicative of the physiological conditions described above. Accordingly, the neonate pod with charger 402 may send these signals to the controller 70 for processing.

Additionally, FIGS. 4A through 4C show example neonate pods with charger having a single charger 406. However, as stated previously, some embodiments of the present disclosure may include multiple chargers, wherein each of the chargers may be configured to convert different forms of energy (e.g., light, heat, and the like) into electrical current.

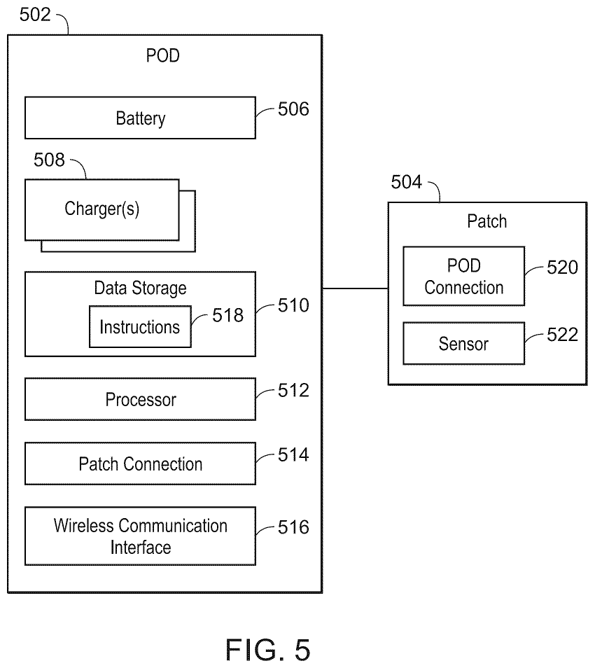

FIG. 5 is a block diagram an example neonate pod with charger (POD) 502 and patch 504 according to one embodiment of the present disclosure. The line connecting the POD 502 with the patch 504 represents the physical connection between the POD 502 and patch 504. Additionally, the POD 502 includes a battery 506, charger 508, data storage 510, processor 512, patch connection 514, and wireless network interface 516. The POD 502 may be similar to the neonate pod with 302, 402 described with respect to FIGS. 3, 4. The battery 506 may be similar to the battery 404 described with respect to FIGS. 4A, 4B, 4C. The charger(s) 508 may be similar to the charger 406 described with respect to FIGS. 4A, 4B, 4C. The data storage 510 may be computer memory or storage device, including volatile memory, such as a random access memory (RAM) device (e.g., static RAM, dynamic RAM, and the like), non-volatile memory, such as a hard disk drive, solid state device (SSD), removable memory cards, optical storage, flash memory devices, and the like. The processor 512 may be a computer processing circuit (e.g., a central processing unit (CPU)) that retrieves and executes programming instructions stored in the memory data storage 510 to perform the functionality described herein. The patch connection 514 may be a physical connection, such as a set of pins, plugs, or sockets configured to connect with a POD connection 518 of the patch 504. In contrast to current neonatal pods where the connection between the pod and patch may include magnets, the patch connection 514 and POD connection 518 may include lighter weight materials. In this way, the POD 502 may represent an improvement over current devices, where the weight of magnets may cause the patient 1 discomfort. According to some embodiments of the present disclosure, the POD connection 518 may be configured to connect with various patch types, e.g., ECG patch, chest patch, extremity patch, temperature patch, respiratory rate patch, fluid loss patch, and the like. The wireless network interface 516 may be similar to the wireless communication interface 408 described with respect to FIGS. 4A, 4B, 4C.

The patch 504 may be similar to the patches described herein, such as the chest patch 300-1, extremity patch 300-2, described with respect to FIG. 3, and the patches 400A, 400B, 400C, described with respect to FIGS. 4A, 4B, 4C. The patch 504 includes the POD connection 518, and a sensor 520. The POD connection 518 may be a physical interface configured to electrically connect with the patch connection 514 of the POD 502, and to provide the measured signals of the sensor 520 for the POD 502. The sensor 520 may represent the configuration of electrodes used to make physiological measurements based on the type of the patch 504.

Additionally, the data storage 510 may include instructions 518 that perform functions as described herein. More specifically, the instructions 518 may be configured to perform pre-processing and/or housekeeping of the signals from the patch 504. Additionally, the instructions 518 may be configured to automatically detect the type of the patch 504. Further, the instructions 518 may be configured to monitor the wireless connection of the wireless communication interface 516. For example, the instructions 518 may determine that the wireless communication interface 516 does not have a connection to a network (e.g., the wireless connection fails). Accordingly, the instructions 518 may be configured to store the data corresponding to the signals from the patch 504 in the data storage 510 until the wireless communication interface 516 can establish a connection to the network. Additionally, the instructions 518 may be configured to operate the display 42, speaker 44, and lights 46 to sound alerts, provide communication network status, and/or prompt the caretaker to monitor the physiological measurements until the communication is restored. Further, once the wireless connection is re-established, the instructions may be configured to send the data that is stored (during the connection failure) over the network to the controller 70.

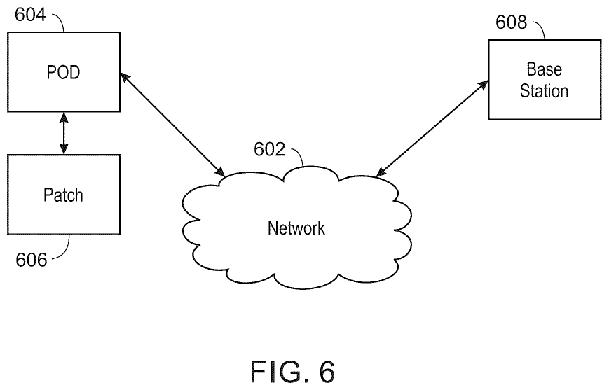

FIG. 6 is a diagram of a system 600 for an example neonate pod with charger (POD) 604 and patch 606 according to one embodiment of the present disclosure. The POD 604 may be similar to the neonate pods with charger 302, 402, and POD 502, respectively described with respect to FIGS. 3, 4A-4C, and 5. Additionally, the patch 606 may be similar to the patches 300-1, 300-2, 400A, 400B, 400C, and 504. The system 600 also includes a network 602 and base station 608. The network 602 may be a computer communication network, or collection of networks, that facilitate communication between the POD 604 and base station 608. In some embodiments of the present disclosure, the network 602 may be a wireless local area network (WLAN), wireless wide area network (WWAN), and the like. The base station 608 may be a device capable of establishing a network connection with a wireless communication interface of the POD 604. Over this connection, the POD 604 may provide sensor data received from the patch 606 to the base station 608. Additionally, the base station 608 may include a processing device, such as the controller 70. Accordingly, the base station 608 may be configured to make determinations about the patient's health based on the data received from the POD 604.

As used herein, the term, mechanism, can encompass hardware, software, firmware, or any suitable combination thereof. In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A neonatal pod with charger, comprising:
a physical connection configured to couple to a neonatal patch that is configured to measure signals from a patient;
a wireless communication interface;
a rechargeable battery; and
a battery charger that is configured to convert ambient radiation into an electrical current, and charge the rechargeable battery using the electrical current.

2. The system of claim 1, wherein the battery charger comprises a solar charger.

3. The system of claim 1, wherein the battery charger comprises a thermal charger.

4. The system of claim 3, wherein the thermal charger converts thermal energy from a body of the patient to electrical current.

5. The neonatal pod with charger of claim 1, comprising:
a processing device; and
a memory device configured to store instructions executable by the processing device to use the wireless communication interface to provide the signals over a network to a base station configured to make determinations about the health of the patient based on the signals.

6. The neonatal pod with charger of claim 5, wherein the instructions are executable by the processing device to:
determine that the wireless communication interface is not connected to the network; and
store the signals in the memory device until the wireless communication interface is re-connected to the network.

7. The neonatal pod with charger of claim 6, comprising an alarm device, and wherein the instructions are executable by the processing device to activate the alarm in response to determining that the wireless communication interface is not connected to the network.

8. The neonatal pod with charger of claim 1, wherein the instructions are executable by the processing device to determine a type of the neonatal patch.

9. The neonatal pod with charger of claim 1, wherein the signals represent a physiological condition of the patient.

10. The neonatal pod with charger of claim 1, wherein the patch is selected from a group consisting of an electrocardiogram patch, a core temperature patch, a peripheral temperature patch, a respiratory rate patch, a pulse rate patch, and a specific percentage of oxygen patch.

11. A system, comprising:
a processing device; and
a memory device configured to store instructions executable by the processing device;
a physical connection configured to couple to a neonatal patch that is configured to measure signals from a patient;
a wireless communication interface;
a rechargeable battery; and
a battery charger that is configured to convert ambient radiation into an electrical current, and charge the rechargeable battery using the electrical current.

12. The system of claim 11, wherein the battery charger comprises a solar charger.

13. The system of claim 11, wherein the battery charger comprises a thermal charger.

14. The system of claim 13, wherein the thermal charger converts thermal energy from a body of the patient to electrical current.

15. The system of claim 11, wherein the instructions are executable by the processor to use the wireless communication interface to provide the signals over a network to a base station configured to make determinations about the health of the patient based on the signals.

16. The system of claim 15, wherein the instructions are executable by the processing device to:
determine that the wireless communication interface is not connected to the network; and
store the signals in the memory device until the wireless communication interface is re-connected to the network.

17. The system of claim 16, comprising an alarm device, and wherein the instructions are executable by the processing device to activate the alarm in response to determining that the wireless communication interface is not connected to the network.

18. The system of claim 11, wherein the instructions are executable by the processing device to determine a type of the neonatal patch.

19. The system of claim 11, wherein the signals represent a physiological condition of the patient.

20. The system of claim 11, wherein the patch is selected from a group consisting of an electrocardiogram patch, a core temperature patch, a peripheral temperature patch, a respiratory rate patch, a pulse rate patch, and a specific percentage of oxygen saturation in the blood patch.
